(19) United States
(12) Patent Application Publication
(10) Pub. No.: US 2022/0072185 A1
(43) Pub. Date: Mar. 10, 2022

(54) SYSTEMS AND METHODS FOR ULTRAVIOLET TREATMENT OF INDOOR CONTAMINANTS

(71) Applicant: Joel H. Miller, Atlantic City, NJ (US)

(72) Inventor: Joel H. Miller, Atlantic City, NJ (US)

(21) Appl. No.: 17/013,283

(22) Filed: Sep. 4, 2020

(51) Int. Cl. A61L 9/20; G01N 33/00; B01D 53/00

(52) U.S. Cl. CPC A61L 9/20; G01N 33/0027; B01D 53/007; B01D 2259/804; A61L 2209/111; A61L 2209/15; A61L 2209/12

(57) ABSTRACT

Enabling systems and methods for ultraviolet treatment of ambient air contaminants are disclosed. One disclosed example computing device includes a housing; a fan, a UV light source configured to emit UV light, and a baffle configured to slow air flow, wherein each is positioned within the housing; and wherein the UV treatment system is configured to: cause a movement of the fan that induces the air flow and moves contaminants through the baffle and around the UV light source; transmit an electrical signal to the UV light source to cause the UV light source to emit UV light in a first frequency range to generate a quantity of an oxidizing agent and in the second frequency range to eliminate or kill contaminants and reduce at least a portion of one or more undesirable byproducts; and expel the treated air out of the housing via the air flow pathway.

### FIELD

The present application generally relates to ultraviolet treatment of indoor contaminants, and more particularly relates to ultraviolet treatment of ambient indoor contaminants in an indoor environment.

### BACKGROUND

The use of air cleaning devices for sanitization and cleaning of an indoor environment is becoming increasingly common. Indoor contaminants, such as noxious gases or undesirable particulates, e.g., dust, bacteria, other contaminants, etc., may be released into an environment through normal activities such as walking or breathing Some types of air cleaning devices create electric fields to oxidize air molecules, which may attract contaminants to them and may then deposit on nearby surfaces, e.g., metal plates in the air cleaning device or on other surfaces within the environment. Thus, oxidized air molecules can physically remove contaminants from the air through electrostatic attraction.

### SUMMARY

Various examples are described for systems and methods for UV treatment of ambient indoor contaminants. One example UV treatment device includes a housing; a fan positioned within the housing, the fan configured to induce air flow into and out of the housing through an air flow pathway; a UV light source positioned within the housing and the air flow pathway, the UV light source configured to emit UV light in a first frequency range from a first portion of the UV light source and UV light in a second frequency range from a second portion of the UV light source, and wherein the first frequency range is different from the second frequency range; a baffle positioned within the housing and within the air flow pathway, the baffle configured to slow the air flow induced by the fan to increase exposure time of the air flow to the UV light source; and wherein the UV treatment system is configured to: cause a movement of the fan that induces the air flow and moves contaminants through the baffle and around the UV light source; transmit an electrical signal to the UV light source to cause the UV light source to emit the UV light in the first frequency range to generate a quantity of an oxidizing agent and in the second frequency range to eliminate or kill contaminants and reduce at least a portion of one or more undesirable byproducts; and expel the treated air out of the housing via the air flow pathway.

One example method includes inducing, by a fan positioned within a housing, an air flow into the housing and through an air flow pathway to an outlet from the housing, wherein: a UV light source is positioned within the housing and the air flow pathway, the UV light source configured to emit UV light into the air flow, and a baffle is positioned within the housing and the air flow pathway, the baffle configured to slow the air flow past the UV light source; emitting, by the UV light source, UV light in a first frequency range to generate a quantity of an oxidizing agent and a second frequency range to eliminate or kill contaminants and reduce at least a portion of one or more undesirable byproducts to treat the air in the air flow, and wherein the first frequency range is different from the second frequency range; and expelling, by the fan, the treated air out of the housing via the air flow pathway and the outlet from the housing.

One example non-transitory computer-readable medium includes program code executable by a processor to cause the processor to: transmit a first signal to control a movement of a fan of a UV treatment system, the UV treatment system comprising: a housing; the fan positioned within the housing, the fan configured to induce air flow into and out of the housing through an air flow pathway; a UV light source positioned within the housing and the air flow pathway, the UV light source configured to emit UV light in a first frequency range from a first portion of the UV light source and UV light in a second frequency range from a second portion of the UV light source, and wherein the first frequency range is different from the second frequency range; and a baffle positioned within the housing and within the air flow pathway, the baffle configured to slow the air flow induced by the fan to increase exposure time of the air flow to the UV light source; and transmit a second signal to cause the UV light source to emit the UV light in the first and second frequency ranges, wherein UV light in the first frequency range is configured to generate a quantity of an oxidizing agent and UV light in the second frequency range is configured to eliminate or kill contaminants and reduce at least a portion of one or more undesirable byproducts to treat the air in the air flow.

These illustrative examples are mentioned not to limit or define the scope of this disclosure, but rather to provide examples to aid understanding thereof. Illustrative examples are discussed in the Detailed Description, which provides further description. Advantages offered by various examples may be further understood by examining this specification.

### BRIEF DESCRIPTION OF THE DRAWINGS

The accompanying drawings, which are incorporated into and constitute a part of this specification, illustrate one or more certain examples and, together with the description of the example, serve to explain the principles and implementations of the certain examples.

FIG. 1 shows an example environment for a system for UV treatment of ambient indoor contaminants.

FIG. 2 shows another example environment for a system for UV treatment of ambient indoor contaminants.

FIG. 3 shows yet another example environment for systems for UV treatment of ambient indoor contaminants.

FIG. 4 shows an example system for UV treatment of ambient indoor contaminants.

FIG. 5 shows another example system for UV treatment of ambient indoor contaminants.

FIG. 6 shows another example system for UV treatment of ambient indoor contaminants.

FIG. 7 shows yet another example system for UV treatment of ambient indoor contaminants.

FIG. 8 shows a graph of test results using a system for UV treatment of ambient indoor contaminants over time.

FIG. 9 shows another graph of test results using a system for UV treatment of ambient indoor contaminants over time.

FIG. 10 shows an example method for UV treatment of ambient indoor contaminants.

FIG. 11 shows another example method for UV treatment of ambient indoor contaminants.

FIG. 12 shows an example computing device for a system for UV treatment of ambient indoor contaminants.

### DETAILED DESCRIPTION

Examples are described herein in the context of systems for UV treatment of ambient indoor contaminants. Those of ordinary skill in the art will realize that the following description is illustrative only and is not intended to be in any way limiting. Reference will now be made in detail to implementations of examples as illustrated in the accompanying drawings. The same reference indicators will be used throughout the drawings and the following description to refer to the same or like items.

In the interest of clarity, not all of the routine features of the examples described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another.

Systems and methods according to this disclosure enable the use of functionality in a system for UV treatment of ambient indoor contaminants. These functionalities are enabled while mitigating or preventing risks associated with traditional air treatment techniques. Certain aspects of this disclosure describe example systems for UV treatment of ambient indoor contaminants that include advantages over existing solutions. For example, some existing systems attempt to disinfect air by forcing air through a filter, which may trap some air contaminants, but, over time, filters can become clogged and less effective, thus requiring frequent, regular maintenance. Further, some UV systems may remove some contaminants air but fail to control the flow of air, which causes a disinfection of fewer contaminants, reducing an overall effectiveness of any UV treatment.

In contrast to existing systems, certain aspects of this disclosure control the flow of air through the UV treatment system, generating highly unstable air molecules by exposing these molecules to multiple wavelengths of UV light that disinfects air by disrupting and/or destroying certain particulate matter. Further, certain aspects of this disclosure includes disinfectant techniques that create air circulation patterns that increase an overall effectiveness of the system over time. For example, certain aspects of this disclosure enable the generation of treated air that is highly molecularly unstable within the device to treat air and surfaces outside the device.

As an illustrative example, an air treatment system induces an air flow into a housing of the air treatment system, e.g., using a centrifugal fan. The air treatment system employs a baffle that slows down the induced air flow and increases a length of time the induced air flow is inside the housing of the air treatment system. The air treatment system uses a UV light source to disinfect air (e.g., from the induced air flow) in the housing. Further, the UV light source creates oxidized air that cleans outside the air treatment system.

In this example, the air treatment system uses the UV light source to produce germicidal UV light. The UV light includes a customized U-shaped tube, which can increase the overall functionality of the system by increasing a quantity of highly molecularly unstable air. For example, the U-shaped tube provides a larger quantity of germicidal UV light within the housing of the air treatment system. And in some examples, the U-shaped tube creates oxidized air that may not require being in a certain proximity to the UV light source for disinfection.

This increased quantity of germicidal UV light creates a large volume of molecularly unstable air that can elimination more indoor contaminants. For example, the U-shaped tube can include a first section that generates an oxidizing agent and a second section that degrades the oxidizing agent. When doing so, the air treatment system degrades a large portion of an oxidizing agent in the air flow, generating treated air. By degrading a large portion of the oxidizing agent, the air treatment system may also reduce an amount of undesirable byproducts. Further, the UV treatment system can use the centrifugal fan to expel the treated air into a surrounding environment.

This illustrative example is given to introduce the reader to the general subject matter discussed herein and the disclosure is not limited to this example. The following sections describe various additional non-limiting examples and examples of systems for UV treatment of ambient indoor contaminants.

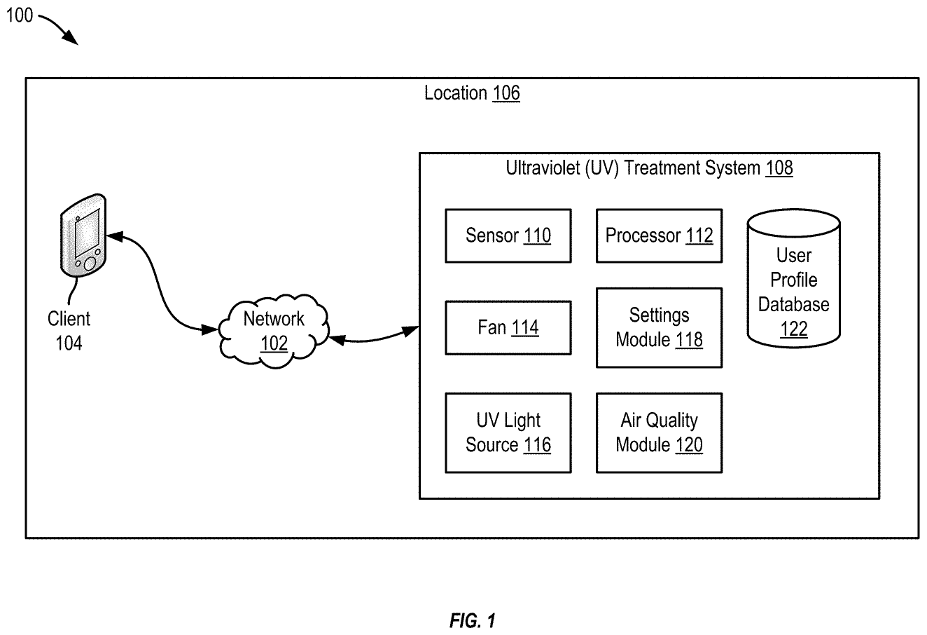

Referring now to FIG. 1, FIG. 1 shows an example environment 100 for a system for UV treatment of ambient indoor contaminants. In this example, the environment 100 includes components located within a location 106, as well as other components located at any suitable location, including remotely from the location 106. Within the location 106 are a client device 104 and a ultraviolet (UV) treatment system 108, which includes a sensor 110, processor 112, fan 114, UV light source 116, settings module 118, air quality module 120, and user profile database 122.

While the example shown in FIG. 1 only includes one client device and one UV treatment system 108, it should be appreciated that the 106 may have more than one client device 104, UV treatment system 108, sensor 110, processor 112, fan 114, UV light source 116, settings module 118, air quality module 120, or user profile database 122. Further, the example UV treatment system 108 shown in location 106 may be communicatively coupled to client devices 104 and/or other computing devices that are remotely located from location 106. For example, a network 102 can be used to allow for remote access to the UV treatment system 108, enabling remote monitoring, settings, and/or other adjustments to the UV treatment system 108.

The UV treatment system 108 may include a housing with one or more components in it. For example, the UV treatment system 108 shown in FIG. 1 includes a sensor 110, processor 112, fan 114, UV light source 116, settings module 118, air quality module 120, and user profile database 122. In some examples, the UV treatment system 108 may include an air flow pathway defined within it (e.g., defined by an air channel). Further, UV treatment system 108 may include a positioning of the UV light source 116 that is placed substantially in or adjacent to the air flow pathway.

In some examples, the UV treatment system 108 may include one or more baffles that can slow an air velocity induced by fan 114 down. At one end of the air flow pathway is an air inlet and at the other end is an air outlet. Air is drawn into the air inlet by the fan 114, traverses through the air flow pathway, across the UV light source 116 and/or baffles, and then flows out of the outlet. As the air flow is slowed by one or more baffles, it can be exposed to different frequency ranges of UV light to eliminate various contaminants in the air flow, creating treated air. This treated air is expelled from the outlet and back into the environment.

The UV treatment system 108 is in communication with one or more client devices 104 via network 102. The network 102 may be any combination of local area networks (“LAN”), wide area networks (“WAN”), e.g., the Internet, etc. that enables electronic communications between the UV treatment system 108, client devices 104, and/or one or more remote computing devices.

The client device 104 may be a cell phone, laptop, personal computer (PC), smartphone, smart watch, tablet, phablet, gaming device, IoT device, server, another computing device, or any other suitable processing device. The client device 104 may include a graphical user interface (GUI), touch surface, a camera, one or more buttons, knobs, or another suitable user input control. In some examples, client device 104 may be wall mounted (e.g., a control panel or touchscreen device). And in some examples, the client device 104 may recognize a user inputs that includes a gesture (e.g., via a camera). The client device 104 can send instructions to the UV treatment system 108 via network 102, e.g., based on a user input.

In this example, the UV treatment system 108 includes a sensor 110. The sensor 110 may include one or more sensors, such as a barometer, carbon dioxide, carbon monoxide, contact, contaminants, chemical, formaldehyde, gas, humidity, light, mold, nitric oxide (NO), nitrogen dioxide (NO2), oxygen, ozone (O3), optical, particulate matter, pollutant, pressure, VOC, smoke, spore, temperature sensor, another sensor, etc. Sensor 110 is in electrical communication with the UV treatment system 108 and is capable of detecting environmental conditions and sending one or more sensor signals that indicate one or more environmental conditions. The sensor 110 may detect environmental conditions such as bacteria, carbon dioxide (CO2), carbon monoxide (CO), combustible gases, dust mites, formaldehyde (HCOH), humidity, mold, motion, natural gas, nitrogen oxide (NO), oxygen (O2), ozone, volatile organic compounds (VOCs), particulate matter, propane gas, radon, smoke, spores, temperature, etc.

The sensor 110 shown in FIG. 1 is integrated in UV treatment system 108. But in some examples, the sensor 110 may be remote from the UV treatment system 108. For example, the sensor 110 may be included in client 104 or located elsewhere in location 106. In some examples, the sensor 110 is remote from the UV treatment system 108, but the sensor 110 may still be communicatively coupled to UV treatment system 108. For instance, the sensor 110 may transmit sensor signals to the UV treatment system 108 continuously or at regularly scheduled (e.g., predetermined) intervals.

In some examples, the sensor 110 may transmit sensor signals to the UV treatment system 108 in response to a request, e.g., from client device 104, UV treatment system 108, a remote computing device, etc. Further, the sensor 110 can generate a sensor signal that indicates a sensed amount of a detected environment condition, e.g., in an indexed value (e.g., air quality index (AQI)), micrograms per cubic meter (e.g., μg/m³), parts per million (ppm), parts per billion (ppb), percentage, etc. In some examples, sensor 110 can transmit sensor signals to client device 104, UV treatment system 108, another remote computing device, etc., or a combination of these.

In this example, the UV treatment system 108 includes a fan 114. The fan 114 may include an axial, centrifugal, exhaust, electrostatic, impeller, multi-directional, oscillating, radial, rotary, solar-powered, static pressure, high static pressure, tangential, tilting, wall-mounted, window-mounted, another type of fan, etc. The fan 114 may include features such as an air inlet, air outlet, blade, blower, chamber, cover, duct, filter, housing, motor, mount, pump, slat, variable speed, other features, etc. In this example, the fan 114 is in electrical communication with the UV treatment system 108 and is capable of creating air flow along a path within UV treatment system 108.

For example, fan 114 can induce air flow by pulling air into the UV treatment system 108 and expelling the air from a housing around UV treatment system 108. In some examples, the UV treatment system 108 sends an electrical signal that causes a movement of the fan 114 that induces air flow and moves contaminants through a housing of the UV treatment system 108. Further, the air flow may move contaminants around various components of the UV treatment system 108, e.g., a baffle, UV light source 116, another component, etc.

In some examples, the UV treatment system 108 may purify air outside the machine (e.g., outside of a housing for the UV treatment system 108). For example, the UV treatment system 108 may use the fan 114 to disinfect air in a predefined space (e.g., a room of a building) by taking in air from the predefined space, generating treated air, and expelling a quantity of the treated air into the predefined space. The predefined space may include a building, chamber, cubicle, dorm, dwelling, home, hotel, structure, warehouse, etc., or a combination of these.

In some examples, the UV treatment system 108 may generate atmospheric radicals (e.g., hydroxyl or hydroperoxyl radicals) in response to an oxidization process caused by the UV light source 116. Further, an overall efficacy of the UV treatment system 108 may be optimized by introducing an amount of air flow into the predefined space. For example, the UV treatment system 108 may generate these atmospheric radicals more efficiently when fresh air (e.g., including additional oxygen and/or hydrogen molecules) are introduced to the predefined space from an outside source, e.g., via a HVAC system.

In some examples, the fan 114 may expel a high quantity of highly unstable air outside of the UV treatment system 108. For instance, the highly unstable air may include one or more oxidizing agents that are capable of acting as a sanitizing agent with germicidal properties outside of the UV treatment system 108. In some examples, the one or more oxidizing agents may include hydroxyl radicals and/or hydroperoxyl radicals. Advantageously, expelling highly unstable air outside of a housing of the UV treatment system 108, e.g., that includes an amount of hydroxyl radicals and/or hydroperoxyl radicals, a smaller overall footprint of the UV treatment system 108 may be achieved. A smaller UV treatment system 108 may provide a lower overall ambient noise level (e.g., by using a smaller fan). Further, by expelling a large volume of highly unstable air outside of the UV treatment system 108, fewer air contaminants, particulate matter, bacteria, viruses, VOCs, etc. may be drawn into a housing of UV treatment system 108 over time because ambient indoor contaminants may be treated before entering UV treatment system 108.

In this example, the UV treatment system 108 also includes a UV light source 116. The UV light source 116 may include one or more of an amalgam UV light bulb, a cold cathode UV light bulb, a hot cathode UV light bulb, high-output quartz, light emitting diode (LED), high-intensity discharge (HID), sodium discharge, another gas-discharge or gas-filled lamp, etc. The UV light source 116 is in electrical communication with the UV treatment system 108 and can provide UV light that creates unstable air molecules.

In some examples, the UV light source 116 may include one or more UV lamps that are positioned such that the overall footprint may be reduced. For instance, the UV light source 116 may be positioned such that an air flow created by the fan 114 moves around substantially all sides of a cylindrically-shaped UV lamp (e.g., a tube). The UV light source 116 generates oxidizing agents, e.g., by recreating gas reactions that may occur naturally in the troposphere. In some examples, the UV light source 116 outputs certain wavelengths that generates ozone. Further, the UV light source 116 can output wavelengths that transform an amount of the generated ozone into one or more oxidizing agents.

For instance, the UV light source 116 can output a predetermined wavelength or range of wavelengths that cause reactions with the amount of generated ozone. In one example, the UV light source 116 can cause a reaction that is represented by the following expression.

O₃+hv→O(¹D)+O₂

Here, h represents Plank's constant, v represents a frequency of a wavelength, and O(¹D) represents an oxygen atom in an excited state. While O₂ (O2) represents approximately 21% of the ambient air in the atmosphere, the excited oxygen atoms O(¹D) may be extremely reactive with various molecules in ambient air.

In some examples, the excited oxygen atoms O(¹D) may produce the one or more oxidizing agents. For example, the excited oxygen atoms O(¹D) may react with an amount of nitrogen (N2) or an amount of humidity (H20) present in ambient air. In one example, the excited oxygen atoms O(¹D) may react with the amount of nitrogen, which is represented by the following expression.

O(¹D)+N₂→O(³P)+N₂

Here, O(³P) represents a yield that includes an extremely reactive form of atomic oxygen. In another example, the excited oxygen atoms O(¹D) may react with the amount of water, which is represented by the following expression.

O(¹D)+H₂O→OH+OH

Here, OH represents a hydroxyl radical and the yield includes two hydroxyl radicals.

In some examples, the UV light source 116 may generate an amount of hydroxyl radicals that is sufficient to create an amount of hydroperoxyls. For instance, the hydroxyl radicals can transform into hydrogen peroxyl radicals through an interaction with one or more gases present in the ambient air. In one example, a hydroxyl radical OH may react with a carbon monoxide molecule, which is represented by the following expression.

OH+CO→CO₂+H

Here, the reaction transforms harmful carbon monoxide CO into carbon dioxide CO₂ and a hydrogen molecule H. Further, the leftover hydrogen molecule H may react with ambient oxygen and another molecule, which is represented by the following expression.

H+O₂+M→HO₂+M

Here, M represents any suitable third molecule, and the reaction yields a hydroperoxyl radical HO₂.

In some examples, the hydroxyl radicals and hydroperoxyl radicals act as oxidizing agents. For instance, a high ionization energy of both hydroxyl radicals and hydroperoxyl radicals may work in tandem to disinfect ambient air. These hydroxyl radicals and hydroperoxyl radicals can safely disassociate VOCs and deactivate germs. Further, the UV treatment system 108 can use the UV light source 116 to introduced hydroxyl radicals and hydroperoxyl radicals to a predefined space. Thus, the UV treatment system 108 may generate oxidizing agents (e.g., hydroxyl radicals and hydroperoxyl radicals) that perform substantially similar functions in an indoor environment as they perform in the troposphere.

Further, an overall efficiency of the UV treatment system 108 may be optimized by employing a greater quantity of smaller or more efficient UV light sources 116. In some examples, the UV light source 116 may include a UV LEDs or a high-output quartz lamp. In one example, the UV light source 116 may include UV LEDs that are mounted or housed within a light tube or another suitable container. In some examples, an efficiency of the UV LEDs may be increase, e.g., by adding an anti-reflective coating to a housing, light tube, container, etc. of the UV LEDs.

In this example, the UV light source 116 includes a gas that is capable of generating UV light when energized. For example, the UV treatment system 108 can use processor 112 to send an electrical signal to a circuit that applies a voltage across the gas, energizing it. The energized gas emits photons of a desired wavelength or range of wavelengths, e.g., within the UV spectrum, that are directed onto air flowing through the system.

The UV light source 116 may include tubes that have one or more shapes, such as a curved, fluted, linear, L-shaped, U-shaped, V-shaped, or another suitable shape, etc. In some examples, the UV light source 116 has multiple different sections that may generate different wavelengths of UV light. For instance, the UV light source 116 may include a first section that is sealed on both ends and a second section that is sealed on both ends. Further, the sections of a tube for the UV light source 116 may be coupled by an adhesive, curing, fusion, melting, another suitable process, or any combination of these. And in some examples, the UV light source 116 may include tubes that have one or more gases.

For example, the UV light source 116 may have one or more tubes that can generate wavelengths of light within a predetermined range. In one example, the UV light source 116 includes a tube that contains a noble gas (e.g., elements belonging to group 18 of the periodic table such as argon (Ar), krypton (Kr), xenon (Xe), etc.) for producing UV light within a range of wavelengths. For instance, the UV light source 116 may include a tube that produces UV-C light within a range of 100-280 nanometers (nm). The UV light source 116 may also include a tube or a portion of a tube that produces UV-A (e.g., 320-400 nm), UV-B (e.g., 280-320 nm), or UV-C (e.g., 200-280 nm) light wavelengths. In some examples, the UV light source 116 may produce wavelengths that include Vacuum UV (VUV) wavelengths, (e.g., having a spectrum of wavelengths that range from 100-300 nm). This UV light may be short-wave UV light that has germicidal effects (e.g., UVGI or VUV wavelengths) and can be absorbed naturally (e.g., by the ozone and/or atmosphere).

In some examples, the UV light source 116 includes a tube that contains one or more gases. For example, the UV light source 116 may be a gas-filled UV lamp that contains one or more gases, plasmas, or coatings associated with one or more gases. In some examples, the UV light source 116 includes a tube that has been treated with one or more gases. In some examples, UV light source 116 includes a tube with one or more elements, e.g., mercury (Hg), sodium (Na), sulfur (S), other metals or nonmetals, etc. Further, some examples may include mixtures of different elements depending on the desired wavelengths of light for a particular application.

For instance, the tube may be a U-shaped tube that includes sectional portions. Each sectional portion of the tube may have a coating with one or more gases. For example, the tube may include a first portion that includes both a first gas and a metal and a second portion having a second gas and the metal. In one example, the first portion of the tube includes a first gas (e.g., argon) and a metal such as mercury (e.g., mercury vapor). In this example, the second portion of the tube includes the metal mercury and a second gas (e.g., neon) that is different from the first gas. This may allow a single tube to emit UV light in different wavelength ranges, enabling different types of decontamination by the same light source.

In some examples, the single tube may contain different gases that can be designed to provide targeted disinfection and deactivation of specific air contaminants (e.g., particular germs, viruses, bacteria, VOCs, etc.). Further, the type of the gases in the single tube may be determined based on desired light frequency ranges that are optimized based on these targeted air contaminants. It should be appreciated that the tube may include any suitable number of sections, and each section may include any suitable gas or combination of gases.

In some examples, the UV light source 116 may be formed in a U-shaped tube. For example, the UV light source 116 may include a U-shaped tube with a sufficient length to allow an amount of air flow (e.g., an air velocity or air volume flow) to traverse the UV light source over an extended period of time, allowing more complete decontamination of the air. In some examples, one or more baffles may be positioned to obstruct air movement, further increasing the amount of time the air is exposed to the UV light, but without requiring the use of a longer UV light source.

In some examples, a positioning of UV light sources 116 may increase an overall efficiency of the UV treatment system 108. For instance, the UV light source 116 may include a U-shaped light tube that includes a first section and a second section that generates longer frequency wavelengths (e.g., 240-280 nm) and shorter frequency wavelengths (e.g., less than 240 nm), respectively. In one example, a longer frequency wavelength range (e.g., 240-280 nm) can be used to kills germs and bacteria. Further, such a longer frequency range may also aid in the decomposition of the ozone, making it more reactive to chemicals (e.g., VOCs) it comes in contact with but hastening the rate at which it reverts to oxygen (e.g., degrades).

In some examples, a remaining amount of oxygen and/or an oxidizing agent may be expelled from the UV treatment system 108 to kill germs and bacteria that are present in the indoor environment outside of the UV treatment system 108. For instance, the oxygen and/or an oxidizing agent (e.g., atomic oxygen, molecular oxygen, hydroxyls, etc.) may interact with contaminants in the ambient air or on surfaces in the indoor environment. In one example, the content of the light tube may be optimized to disinfect a VOC (e.g., formaldehyde) that is prevalent in certain areas or environments. In some examples, a number of light tubes may be increased in a housing that includes a larger overall footprint. In such a case, an increased number of light tubes may be used to treat air for larger spaces (e.g., in an industrial or animal husbandry environment).

The U-shaped tube may include a predetermined minimum length (e.g., 500 cm). The UV light source 116 may include a U-shaped tube that is equivalent to a 1 meter (m) straight tube (e.g., end-to-end). In some examples, one or more shorter tubes may be used in addition to a U-shaped tube. In other examples, one or more shorter tubes may be used instead of a U-shaped tube. However, in some examples, it may be advantageous to use a U-shaped tube instead of two or more shorter, straight tubes to reduce costs, e.g., in manufacturing. Further, using a U-shaped tube instead of two or more tubes may reduce an overall size or footprint of UV light source 116, or reduce the complexity of the device by reducing the number of electrical contacts, the amount of wiring, etc.

In some examples, a U-shaped optic can enable multiple frequencies to be combined in a space saving form. For instance, UV light wavelengths shorter than 240 nm create ozone via photolysis of oxygen molecules in the air, while UV light wavelengths between 240-280 nm will destroy ozone via photolysis of the ozone molecule. In this example, in order to provide sufficient indoor air purification, the UV light source 116, the U-shaped tube may be structured for a higher wavelength UV light that is sufficient to ameliorate excess ozone created by shorter wavelength UV light. The UV light source 116 may include a tube that is sufficiently longer to decrease an overall volumetric amount of air required to ameliorate excess ozone.

In some examples, an overall efficacy of the UV treatment system 108 may be increased by using a longer U-shaped tube, while making the indoor environment more comfortable and safe in comparison to existing indoor air treatment systems. In some examples, a longer, U-shaped tube may ensure a sufficient amount of a UV dose is exposed to air traversing the UV treatment system 108. For instance, a sufficient amount of air flow traversing an air flow pathway through the UV treatment system 108 may absorb a larger portion of germicidal UV energy among a microbial population. Thus, the UV treatment system 108 provides (e.g., via fan 114) an amount of air flow that enables UV doses from the UV light source 116 to eradicate contaminants.

In this example, the first and second sections can be positioned such that longer frequency wavelengths are generated along an air flow pathway and is positionally closer to an air outlet than the air inlet. By generating longer frequency wavelengths along the air flow pathway after the shorter frequency wavelengths, an amount of ozone generated by the shorter frequency light can be substantially eliminated by the longer frequency light. And in doing so, the UV treatment system may reduce the amount of ozone, while generating an amount of positive byproducts (e.g., the oxidized hydroxyl radicals and hydroperoxyl radicals) and their associated disinfecting properties intact. In some examples, the UV treatment system 108 may include a housing that has an internal reflective surface that further optimizes a delivery of UV doses from the UV light source 116. For example, the housing may include one or more internal reflective surfaces (e.g., a metallic surface) that reflects UV light from sections of the tube for the UV light source 116 within the housing.

In some examples, the UV light source 116 may include one or more frequency wavelengths or ranges of frequency wavelengths that are designed to target specific air contaminants. For instance, the UV light source 116 may target a particular type of air contaminants. In some examples, the UV light source 116 includes wavelengths that kills, destroys, disrupts or otherwise eliminates a type of bacteria, microorganism, VOC, gas, etc. For example, the UV light source 116 may include a wavelength range (e.g., 240-280 nm) that is designed for certain germicidal applications. In another example, the UV light source 116 may include a wavelength range (e.g., 180-240 nm) that is designed for certain VOCs.

In some examples, first and second gases may be apportioned with sections of the tube to produce particular a range of UV light frequencies to target a certain type of VOC or germ. For instance, the first portion of the tube may have a shorter overall length that targets a first type of VOC or germ, while the section portion of the tube may be longer and targets a second type of VOC or germ. In some examples, the shorter section may correspond to a less prevalent type of VOC or germ, while the longer section corresponds to a more prevalent type of VOC or germ. Further, the type of VOC or germ may be based on a category, classification, composition, size, shape, solubility, species, or any other suitable type of VOC or germ.

In some examples, the UV treatment system 108 may include one or more filters. For instance, the UV treatment system 108 may include an electrostatic filter. The electrostatic filter may reduce an amount of particulate matter that is present within a housing of the UV treatment system. In other examples, the UV treatment system 108 may also include a HEPA filter, for example, to filter particulate matter of a certain size. In one example, the type of particulate matter is based on a size, e.g., coarse particulate matter (PM₁₀), fine particulate matter (PM_{2.5}), or ultrafine particulate matter (PM_{0.1}). Further, the length of the second portion of the tube may be an order of magnitude greater than the length of the first portion. By using a shorter first section, an amount of undesirable byproducts (e.g., ozone) may be reduced, while a longer second section may ensure a reduction of those undesirable byproducts before air is expelled into the predefined space.

In some examples, the UV treatment system 108 can use sensor signals from sensor 110 to control one or more operations. For example, the UV treatment system 108 can use processor 112 to execute settings module 118 or air quality module 120 to adjust one or more settings based on a detected amount of particulate matter. In one, the UV treatment system 108 may receive a sensor signal from sensor 110 that indicates an amount of a VOC present in the ambient air for location 106. In this example, the UV treatment system 108 can use air quality module 120 to determine the sensor signal in location 106 includes an undesirable level of the VOC (e.g., formaldehyde). In response, the air quality module 120 can send instructions to the settings module 118 to adjust one or more controls of UV treatment system 108.

For example, the air quality module 120 may send instructions to settings module 118 that indicate an on/off switch, duty cycle, speed, directionality, etc. of a fan 114 should be changed. In some examples, the instructions may include a change to an on/off switch, duty cycle, brightness, etc. of the UV light source 116. In some examples, the air quality module 120 can monitor sensor signals from sensor 110 and generate a report based on sensed environmental conditions. For instance, the air quality module 120 may generate reports that include one or more environmental conditions, system statuses, runtime, uptime, usage, etc. Further, air quality module 120 can send these reports to client device 104, UV treatment system 108, a remote computing device, a combination of these, etc.

In some examples, the UV treatment system 108 is monitored remotely. For instance, UV treatment system 108 may include remote access and/or monitoring via network 102. In one example, the UV treatment system 108 may be located in a school that may be monitored by a remote computing device operated by a school system associated with the school or a third party service. In some examples, UV treatment system 108 may be one of a network of UV treatment systems 108, e.g., operating in the school or school system. In some examples, allowing remote access and monitoring of UV treatment systems 108 may be more efficient, e.g., enabling monitoring, maintenance, and/or operations to be performed by a third party service provider. While this example describes a network of UV treatment systems 108 in a school or school system, it should be appreciated that a network of UV treatment systems 108 may also operate in a business, campus, complex, development, office park, or any other suitable networked locations.

In some examples, the UV treatment system 108 may be part of a home automation or security system. For instance, UV treatment system 108 may include or be in communication with one or more additional sensors or computing devices that can control an operation of the UV treatment system 108. In one example, the UV treatment system 108 may be in electrical communication, e.g., via network 102 with an IoT device for a home automation system (e.g., a smart hub, control panel, touchscreen device, etc.).

In this example, the UV treatment system 108 is associated with the home automation system, which may include a motion sensor that can detect movements in a predetermined space associated with the UV treatment sensor. The motion sensor may send a motion sensor signal to the IoT device that indicates one or more users are present in the predetermined space. In response, the IoT device may send instructions to the UV treatment system 108. For instance, the IoT device can send instructions to the settings module 118 of the UV treatment system 108 that activates or deactivates the UV treatment system 108.

In some examples, the home automation system may include one or more sensors that measure an air quality level. Further, the IoT device of the home automation system can report these measurements to the air quality module 120 of the UV treatment system 108, e.g., by transmitting a sensor signal from the one or more sensors. In additional or alternative embodiments, the IoT device of the home automation system can send instructions to the settings module 118 to turn the UV treatment system 108 on or off based on the sensed air quality level. In some examples, the instructions sent to the settings module 118 may be based on a presence or a level of a specific air contaminant, such as formaldehyde, CO2 or ozone. And in some examples, the instructions sent to the settings module 118 based on the sensor signal may include one or more adjustments to the UV treatment system 108 (e.g., an amount of air flow, air circulation pattern, blower or fan speed, directionality, duty cycle, humidity level, temperature, etc.).

In some examples, the UV treatment system 108 may include additional integrated counters and/or sensors that can report information to the home automation system. For example, the UV treatment system 108 may execute the air quality module 120, which can receive electrical signals from the counters or sensors. The air quality module 120 can report the counter signals and/or sensor signals to the integrated home automation system. In response, the IoT device of the home automation system may send a notification to a user. In one example, the IoT device may send a notification to a user via client 104 that advises the user to replace or clean a feature of the UV treatment system 108.

For example, a counter of the UV treatment system 108 may send an electrical signal to the air quality module 120 after a certain amount of hours of air treatment (e.g., runtime). The air quality module 120 may determine that a dust filter at an air inlet should be checked or replaced to keep device clean based on the runtime. In some examples, the air quality module 120 can send a notification indicating the dust filter should be checked or replaced to the IoT device, client device 104, a user interface device of the UV treatment system, another remote computing device, etc., or a combination of these. Similarly, the air quality module 120 may determine and report additional maintenance information, such as a light tube that should be checked, cleaned, and/or replaced.

In some examples, the reported information may be based on a predetermined amount of on time. But in some examples, the reported information may be based on one or more sensor signals, e.g., that may indicate an element of or function associated with the UV treatment system 108 was operating sub-optimally or had reached an expected lifetime. In some examples, the home automation system may provide a notification or an alert that a part such as a fan, ballast or light tube should be replaced based on a counter or sensor signal.

In some examples, the home automation system may include one or more integrated user controls. The user controls provide user-selectable options to customize one or more operating conditions of the UV treatment system 108. For instance, user controls may include timer settings that can cause the UV treatment system 108 to run or not run at certain times of the day. Further, the user controls may enable a user to set a threshold level for one or more sensor signals (e.g., an atmospheric reading). In response to a sensor signal indicating a user-selected gas concentration above a certain level, the IoT device of the home automation system can send instructions to the settings module 118 to turn the UV treatment system 108 on/off or adjust any of the settings described herein.

In this example, the UV treatment system 108 includes a user profile database 122. The user profile database 122 is integrated within the UV treatment system 108, however, in other examples, the user profile database 122 may be located remotely from the UV treatment system 108. The user profile database 122 may include one or more user profiles that are capable of storing user preferences for the UV treatment system 108. For example, the user profile database 122 may include user profiles that include one or more user preferences such as an air flow, air circulation pattern, alarm, alert, AQI value, authentication tool, automated control, blower or fan speed, directionality, duty cycle, environmental condition, filter controls or replacement, humidity, lighting, noise level, notification, occupancy, on/off control, room setting, setup, temperature, timer, unit of measurement, ventilation, volume control, another environmental condition, etc. In some examples, the user profile database 122 may include user profiles associated with one or more remote computing devices, such as the home automation system described above.

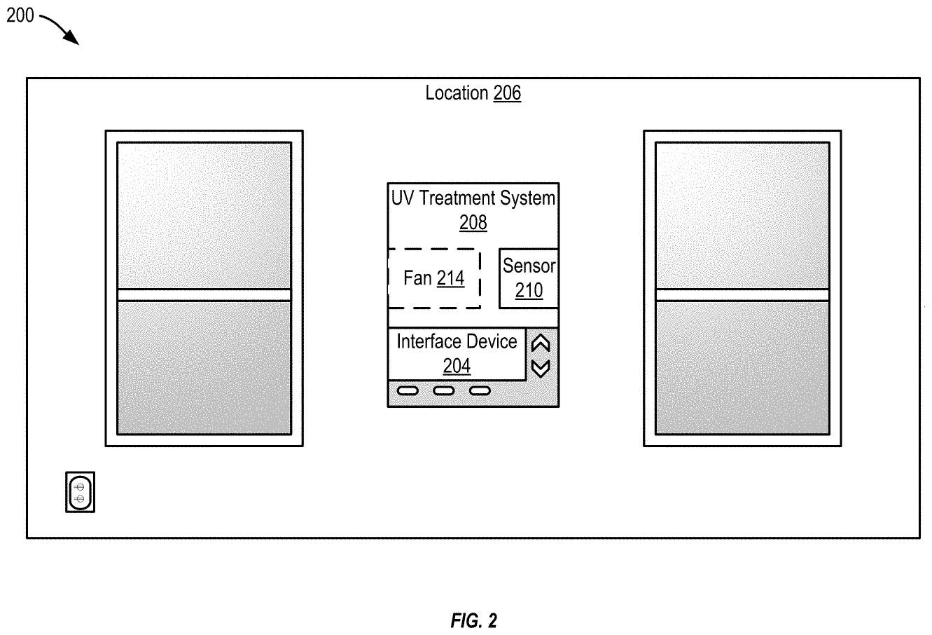

Referring now to FIG. 2, FIG. 2 shows an example environment 200 for a system enabling ultraviolet treatment of ambient indoor contaminants. In this example, the environment 200 includes a location 206 having components of a UV treatment system 208 coupled to and located within a housing for components that include an interface device 204, sensor 210, and fan 214. Similar to client device 104, interface device 204 is communicatively coupled to UV treatment system 208. In this example, UV treatment system 208, sensor 210, and fan 214 are substantially similar to UV treatment system 108, sensor 110, and fan 114, respectively, and may include substantially similar capabilities as described above with regard to like components in FIG. 1. Additionally, UV treatment system 208 may be in communication with client devices or other remote computing devices.

In this example, the environment 200 shows a room in a building that includes a wall mounted UV treatment system 208 that is mounted between two windows. The wall mounted UV treatment system 208 includes a horizontally mounted fan 214 that induces an air flow from a substantially leftward side and expels treated air out of the rightward side. The UV treatment system 208 includes sensor 210, which may include any sensor described herein. In some examples, UV treatment system 208 can automatically adjust a setting based on an ambient environmental condition of the room based on a sensor signal from sensor 210.

In this example, UV treatment system 208 also includes an integrated interface device 204. The interface device 204 may include any of the client devices 104 described in FIG. 1. In one example, interface device 204 includes a control panel, which may be integrated with a remote computing device. Further, the interface device 204 may receive instructions associated with a remote computing device for a home security, home automation, school, or another monitoring system.

The interface device 204 also includes a GUI and can receive touch inputs. In some examples, the interface device 204 may include one or more physical buttons or a touch surface. For instance, the interface device 204 may receive touch inputs for one or more user settings, which may include any of the settings described herein. In some examples, interface device 204 includes a camera that can recognize a user input in a form of a gesture (e.g., a hand movement or swipe). The interface device 204 can control the UV treatment system 208 based on these user inputs.

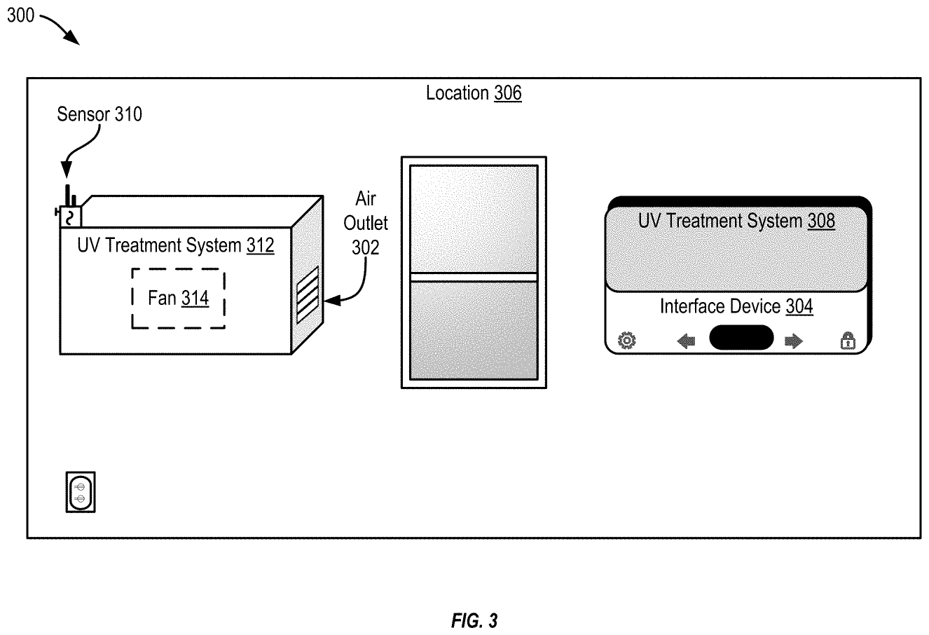

Referring now to FIG. 3, FIG. 3 shows an example environment 300 for systems enabling UV treatment of ambient indoor contaminants. In this example, the environment 300 includes two UV treatment systems 308, 312. UV treatment systems 308, 312, interface device 304, sensor 310, and fan 314 are substantially similar to UV treatment systems 108, 208, interface device 204, sensors 110, 210, and fans 114, 214, respectively, and may include substantially similar capabilities as described above with regard to like components in FIGS. 1 and 2. Further, as described above, UV treatment systems 308, 312 may be in communication with client devices or other remote computing devices.

In the example environment 300, a first UV treatment system 308 and a second UV treatment system 312 are shown as wall-mounted units that may have different aesthetic features. In some examples, the environment 300 may be a substantially larger room (e.g., as measured by volume such as m³) than other examples. For examples, the environment 300 may be an open warehouse space, which is typically volumetrically larger than some rooms, e.g., in residential home. While the two UV treatment systems 308, 312 show a fewer number of components, it should be appreciated that each may include any of the capabilities described above with regard to FIGS. 1 and 2.

For instance, the first UV treatment system 308 may have a slimmer overall design profile, which may be useful for smaller spaces. The second UV treatment system 312 has a greater depth and thicker overall design profile, which may enable a larger fan 314 to move more air through the UV treatment system 312 and expel a greater amount of treated air into a larger space than a smaller design. Further, the second UV treatment system 312 also includes a top-mounted sensor 310, which differs in positioning from sensors 110, 210, and may be positioned accordingly based on a targeted type of contaminant. For instance, some sensors 310 positioned with a minimum a relative height from a floor level may provide increased sensitivity to a sensed amount of a contaminant, such as carbon dioxide, carbon monoxide, hydrogen, nitrogen dioxide, methane, etc.

In this example, the first UV treatment system 308 includes an interface device 304 that is similar to interface device 204. Further, interface device 304 includes a display between left and right arrow buttons, which may be physical buttons or touch surfaces. In some examples, the display may be a touchscreen. In this example, interface device 304 also includes a leftmost settings button.

In some examples, the leftmost settings button of the interface device 304 may be used to display one or more user selectable settings. These user settings can allow the user to change any of the settings for the first UV treatment system 308 described herein. Further, in some examples, the settings button of the interface device 304 may also be used to change one or more settings for a networked computing device. In one example, a user can adjust a setting for the second UV treatment system 312 via a menu option. For instance, a button press of the settings button of the interface device 304 may cause the display to present a GUI menu option corresponding to an adjustable setting of the UV treatment system 312.

In some examples, GUI menu options for the settings button of the interface device 304 may include a subset of all available settings for the second UV treatment system 312. Further, remotely modifying a setting of the second UV treatment system 312 may require an authentication of the user. For example, the display may include a GUI prompt to enter a personal identification number (PIN), username, password, code, biometric data, multifactor information, etc. Once authenticated, the user may be able to adjust settings for the first UV treatment system 308, the second UV treatment system 312, a remote computing device, etc.

In this example, the interface device 304 also includes a rightmost lock button. In some examples, pressing the rightmost lock button of the interface device 304 may wake up the display or prompt a user for authentication information. Further, the lock button may be used by a user to lock one or more settings and/or controls for the interface device 304 or the first UV treatment system 308. In some examples, the lock button of the interface device 304 may restrict access to the first UV treatment system 308, the second UV treatment system 312, a remote computing device, etc., or a combination of these.

The example environment 300 shows the second UV treatment system 312 that includes an air outlet 302. In some examples, the air outlet 302 may include an aperture, diffuser, filter, grill, louvered opening, slot, vent, etc. And while the air outlet 302 is shown as being substantially rectangular, it should be appreciated that the air outlet 302 may be linear, round, oval, square, etc. The air outlet 302 may be positioned to expel treated air in various circulation patterns and/or directions. In some examples, the air outlet 302 may be positioned to discharge treated air in a horizontal, vertical, spreading, non-spreading, or another suitable manner.

In some examples, a size of the air outlet 302 may be determined in proportion with a volumetric size of a defined space. Further, the air outlet 302 may be limited to a predetermined size to reduce an overall ambient noise level. For instance, the size of the air outlet 302 may be determined based on a desired threshold value of ambient noise (e.g., in decibels (dB)). In one example, the size of the air outlet 302 is predetermined such that an amount of treated air is expelled with a sufficient air velocity to travel a predetermined distance, while also maintaining an ambient noise level below the threshold value (e.g., in dB).

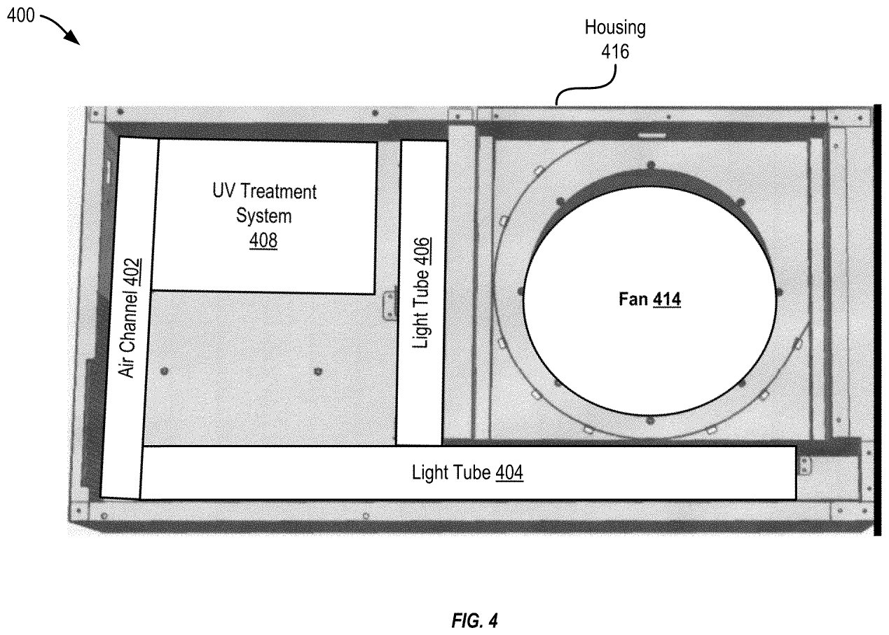

Referring now to FIG. 4, FIG. 4 shows an example 400 of a system for UV treatment of ambient indoor contaminants. The example 400 includes a UV treatment system 408 and fan 414, which are substantially similar to UV treatment systems 108, 208, 308, 312 and fans 114, 214, and 314, respectively, and may include substantially similar capabilities as described above with regard to like components with regard to FIGS. 1-3. However, the example 400 shows a top down view of the UV treatment system 408 that includes an air channel 402, light tube 404, an optional light tube 406, and a housing 416.

The UV treatment system 408 includes the air channel 402, which provides an air flow pathway for air entering the UV treatment system 408, e.g., that is induced by the fan 414. The air channel 402 may be coupled to or integrated in the housing 416. For instance, the air channel 402 may include one or more pipes, side walls, tubes, or any other suitable structure. Further, the air channel 402 may be constructed using an anodized, fiberglass, glass, polymer, plastic, metal, non-conductive, non-magnetic, or another suitable material.

In this example, the air channel 402 directs the air flow along a pathway such that the air passes over or around the light tube 404. The light tube 404 may be any suitable tube described herein. For example, the light tube 404 may be an enclosure containing one or more gases, chemicals, or other treatments generally as described above with respect to FIGS. 1-3. Further, the light tube 404 may be a part of a light source such as UV light source 116. In some examples, the system 400 may include a single light tube 404 that has more than one section and is capable of providing UV light frequencies in more than one frequency range. Further, using a single light tube 404 may reduce costs.

In this example, the system 400 also shows an optional light tube 406. The optional light tube 406 is shown as being adjacent to and perpendicular to the light tube 404. In some examples, the system 400 that employs both light tubes 404 and 406 may disinfect more air contaminants due to the greater length of available light sources and the larger volume of air flow exposed to the emitted UV light.

The example system 400 also shows housing 416, mentioned above. In some examples, the housing 416 may include one or more materials such as fiberglass, glass, polymer, plastic, metal, or any other suitable material. For example, housing 416 may be a metal housing that includes an aluminum chamber. In this example, housing 416 includes anodized aluminum that has an internal reflective surface. In some examples, housing 416 may include one or more internal reflective surfaces that reflect UV light from light tubes 404, 406 in the housing.

The internal reflective surfaces can increase an amount of reflected UV light that boosts an overall efficiency of the system 400. This reflected UV light can minimize an amount of UV energy loss by exposing more air in the air flow pathway to germicidal UV light. Such efficiency gains may require a reduced overall length of light tubes 404, 406. For instance, reflective surfaces of housing 416 can enable shorter light tubes 404, 406 to achieve a desired or optimal volume of treated air that is expelled over time (e.g., in CFM). In some examples, utilizing shorter light tubes 404, 406 to achieve a desired volume of treated air may result in lower power consumption and a more compact size of the example system 400.

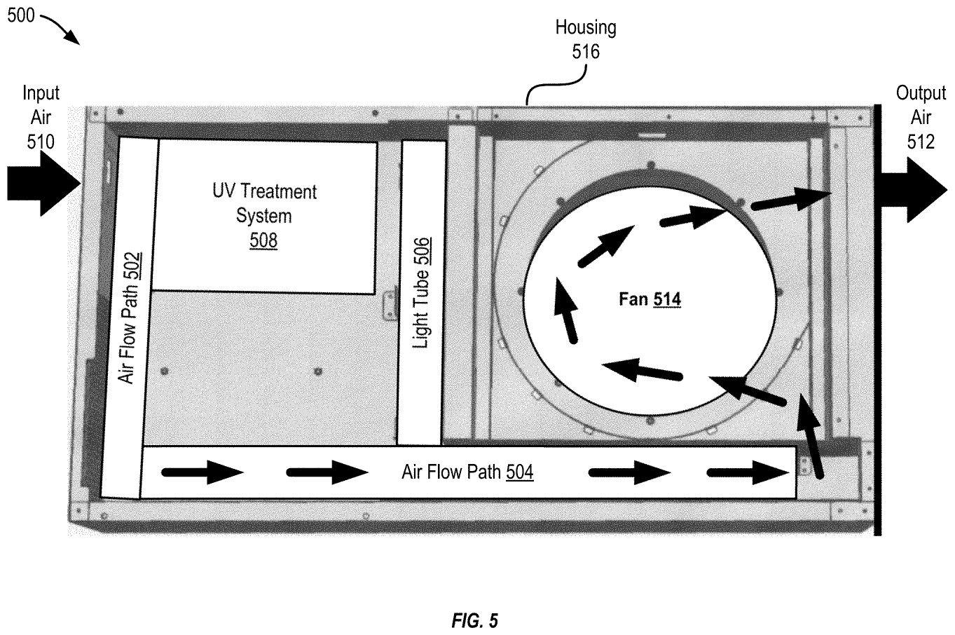

Referring now to FIG. 5, FIG. 5 shows an example 500 of a system for UV treatment of ambient indoor contaminants. The example 500 includes a UV treatment system 508, light tube 506, and fan 514, each of which may be substantially similar to UV treatment systems 108, 208, 308, 312, 408, light tubes 404, 406, and fans 114, 214, 314, and 414, described above with regard to FIGS. 1-4. The UV treatment system 508, light tube 506, and fan 514 may include substantially similar capabilities as described above with regard to like components with regard to FIGS. 1-4. In this example, system 500 shows a top down view that includes an air flow paths 502 and 504. Additionally, input air 510 travels along a pathway corresponding to air flow paths 502, 504.

In this example, the fan 514 induces an air flow. For example, fan 514 can induce an air flow that causes the input air 510 to traverse air flow paths 502, 504, passing light tube 506. In doing so, the input air 510 includes air molecules that traverse air flow paths 502, 504. Air molecules from the input air 510 may be exposed to the UV light created by light tube 506 according to any of the techniques described herein.

The traversal of air flow paths 502, 504 by the input air 510 eliminates air contaminants. In some examples, the air flow paths 502, 504 may be sufficiently long that the input air 510 is exposed to a desired amount of UV energy produced by light tube 506. For instance, air flow paths 502, 504 are shown as including a substantially perpendicular relationship, which may elongate an amount of time that is required for the input air 510 to traverse an overall length of example system 500. In some examples, the air flow paths 502, 504 may include pathways that are non-linear, angular, curved, circuitous, multi-directional, etc., or a combination of these.

In some examples, the fan 514 may be a centrifugal fan that pulls input air 510 into one or more chambers adjacent to air flow paths 502, 504 and over the light tube 506. The input air 510 may continue along air flow paths 502, 504, passing through fan 514, before being expelled as output air 512. Output air 512 can be on the side, top or bottom of the housing 516, depending on the location, type and placement of fan 514. In some examples, fan 514 may be a centrifugal fan that can pull input air 510 into and over light tube 506. But in some examples, the fan 514 may be a centrifugal fan that pulls input air 510 into a chamber, e.g., positioned along or after air flow paths 502, 504, that includes a mounted light tube 506. For instance, the fan 514 may be positioned to direct an air flow through a chamber enclosing the light tube 506, e.g., by pulling air through a top of the housing 516 and expelling air through a bottom of the housing 516. The treated air molecules generated during the traversal of air flow paths 502, 504 is output air 512, which can be expelled from the housing 516, e.g., by the fan 514.

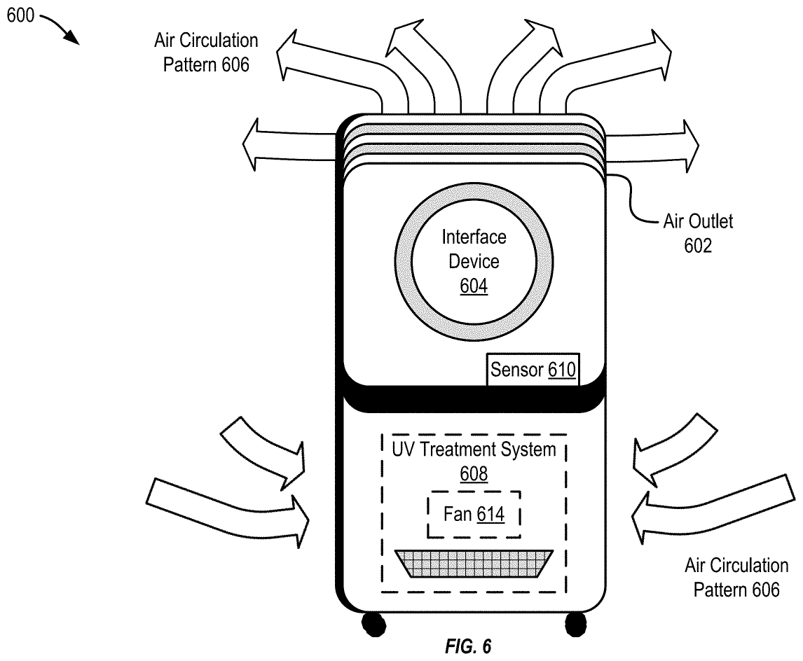

Referring now to FIG. 6, FIG. 6 shows an example system 600 for UV treatment of ambient indoor contaminants. The system 600 includes a UV treatment system 608, interface device 604, sensor 610, and fan 614. The UV treatment system 608 and fan 614 are substantially similar to UV treatment systems 108, 208, 308, 312, 408, 508, interface devices 204, 304, sensors 110, 210, 310, and fans 114, 214, 314, 414, and 514 and include substantially similar capabilities as described above with regard to like components with regard to FIGS. 1-5. But in this example, the example system 600 is a standing unit that produces an air circulation pattern 606, which is controllable by interface device 704 and/or sensor 710.

The UV treatment system 608 can optimize an air circulation pattern 606. For example, the UV treatment system 608 can generate the air circulation pattern 606 such that an amount of air flow reaches all corners of a predefined space to be treated. The circulation pattern 606 includes an optimized air flow that provides measurable improvements to ambient air quality in the predefined space.

For instance, the air circulation pattern 606 may improve air treatment results by creating a cyclical air flow pathway. The cyclical air flow pathway of air circulation pattern 606 may have a sufficient air flow velocity and direction to push air in a predetermined trajectory or over a predetermined distance. In this example, the air circulation pattern 606 shows treated air is expelled by UV treatment system 608 in a vertically-ascending manner. For instance, the treated air that is expelled may include a circulation pattern 606 with one or more directional vectors. In some examples, these vectors may include one or more directions, which may include vectors in substantially 360 degrees of circumferential directions in a horizontal plane that is substantially parallel to a surface beneath the UV treatment system 108. Further, in some examples, the expelled treated air may include vertical, non-horizontal, or any other suitable directionality in three dimensions.

In this example, the air circulation pattern 606 shows the ambient air, e.g., in the predefined space, is cyclically drawn into UV treatment system 608. For instance, the fan 614 shown in UV treatment system 608 is positioned in a lower portion of the UV treatment system 608. This relative positioning of fan 614 enables the fan 614 to induce air flow into the upright, standing UV treatment system 608. The induced air flow by fan 614 completes the cyclical air circulation pattern 606.

In some examples, the UV treatment system 608 may optimize the air circulation pattern 606 based on one or more user settings. For example, a user may control one or more settings for the air circulation pattern 606 via interface device 604. In some examples, the user may input one or more settings via the interface device 604 for an amount of air flow, a type of air circulation pattern 606, blower or fan speed, directionality, duty cycle, etc. In some examples, the UV treatment system 608 may be a part of a larger system (e.g., a HVAC or home automation system), which may allow the user to input one or more user settings for the larger system, such as a desired humidity level or temperature.

In one example, the user can adjust the type of air circulation pattern 606, which may include a cross ventilation, directional, horizontal, vertical, intermittent, oscillatory, randomized, or another suitable pattern, etc. For instance, a cross ventilation pattern may include may include an air flow exiting air outlet 602 in a substantially horizontal and crosswise or X-pattern. Further, such a crosswise or X-pattern may include an air flow that intermittently switches between a leftmost and rightmost side of the UV treatment system 608.

In some examples, the crosswise or X-pattern may be created by two or more UV treatment systems 608. For example, similar to the UV treatment systems 308, 312 described above with regard to FIG. 3, two or more directionally opposing UV treatment systems 608 may create a crosswise or X-pattern based on a crossing of an air flow vector expelled from each. Further, this crosswise or X-pattern may cross in one or more three dimensional planes.

In some examples, the UV treatment system 608 can optimize air circulation by adjusting an amount of air flow in the air circulation pattern 606 based on a desired air quality level. For example, UV treatment system 608 can use an algorithm that determines an optimal amount of air flow in the air circulation pattern 606 to achieve a desired air quality level. In this example, the UV treatment system 608 may use a sensed amount of contaminants that remain in the ambient air (e.g., based on a sensor signal from a sensor such as sensors 110, 210, 310).

In some examples, the UV treatment system 608 may use a desired ambient noise level for the predefined space to determine the optimal amount of air flow in the air circulation pattern 606 (e.g., using a microphone, ambient noise sensor, sound level meter, another audio device, etc.). Further, UV treatment system 608 may determine the optimal amount of air flow in the air circulation pattern 606 based on a desired air quality level, ambient noise level, power consumption, duty cycle, default setting, user preference, etc., or a combination of these.

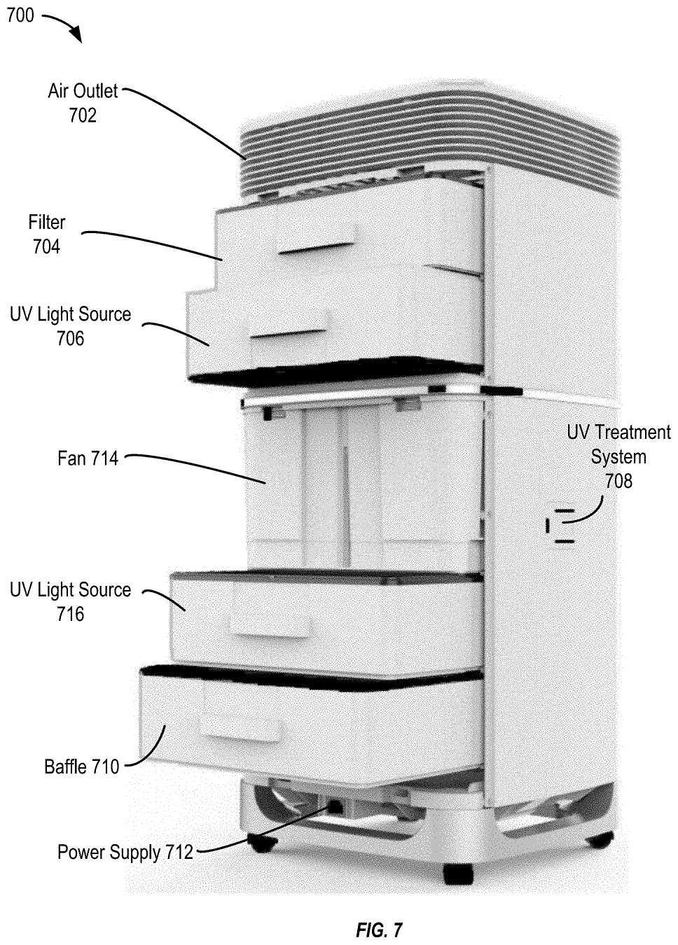

Referring now to FIG. 7, FIG. 7 shows an example system 700 for UV treatment of ambient indoor contaminants. The system 700 includes a UV treatment system 708, air outlet 702, UV light sources 706, 716, and fan 714, which are substantially similar to UV treatment systems 108, 208, 308, 312, 408, 508, 608, 708, air outlet 302, UV light source 116, and fans 114, 214, 314, 414, 514, 614, and 714 and may include substantially similar capabilities as discussed above with regard to FIGS. 1-7. In this example, system 700 is a standing unit that shows one example of relative positioning for the components mentioned above, as well as a filter 704, baffle 710, and power supply 712.

The example system 700 shows a substantially modular design for a floor standing unit according to certain aspects. For example, the modular system 700 includes an air outlet that is integrated with a housing of the system 700. Further, the modular system 700 includes a removable door is integrated into the housing. The removable door may be used to access one or more components and/or computing devices for the UV treatment system 708. The modular system 700 also includes several drawers, e.g., each of which house a filter 704, UV light sources 706, 716, baffle 710, and fan 714. In this example, the power supply 712 is coupled to the housing of the modular system 700 and positioned beneath the drawers. It should be appreciated that, in some embodiments, a number of, type of, order of, and arrangement of the drawers and the components described above may be altered.

For example, the modular system 700 may include any suitable number of air outlets 702, UV light sources 706, 716, baffles 710, power supplies 712, and fans 714. Further, each of the air outlets 702, UV light sources 706, 716, baffles 710, power supplies 712, and fans 714 may be any suitable type described herein. And each of the air outlets 702, UV light sources 706, 716, baffles 710, power supplies 712, and fans 714 may be rearranged. In some examples, the modular system 700 may be integrated within a larger system, such as a HVAC system. Further, the example system 700 may be used in combination with other features such as air conditioning, odor reduction, air particulate filtration or reduction, etc., or a combination of these.

As described above, with respect to FIG. 6, the UV treatment system 708 can create an air circulation pattern for a predefined space. In this example, the UV treatment system 708 does so by using the fan 714 to pull air into the housing through an air inlet. The air inlet may be positioned near the lowermost portion of the upright system 700. For instance, the air inlet may positioned below baffle 710. In one example, the air inlet may be positioned between the baffle 710 and the power supply 712. The baffle 710 can slow an air flow induced by fan 714.

For example, the baffle 710 may be a blanking, deresonating, detuning, disc and doughnut, impingement, longitudinal flow, orifice, segmental (e.g., single or double segmental), support, or any other suitable type of baffle, etc. Further, baffle 710 may be constructed of one or more materials such as aluminum, chrome, steel, stainless steel, titanium, silicone, neoprene, plastic, polymer, polystyrene, or another suitable material. In some examples, the baffle 710 slows the induced air flow caused by the fan 714, reducing air velocity within UV treatment system 708.

In some examples, a reduced air velocity may provide an increased amount of contact time that the induced air flow is exposed to germicidal UV light produced by UV light sources 706, 716. Thus, as the induced air progresses through the UV treatment system 708, rising vertically past the baffle 710, multiple UV wavelengths that are produced by UV light sources 706, 716 may more thoroughly sanitize and/or disinfect a volume of air in the air flow pathway.

In some examples, one or more baffles 710 may be positioned such that an air flow pathway is created that directs around the UV light sources 706, 716 (as opposed to pushing air through the UV light sources 706, 716). In additional or alternative embodiments, a sufficient number of baffles 710 and/or other methods to increase contact time (e.g., an air channel or otherwise defined air flow pathway) may sufficiently reduce air velocity to allow fan 714 to push air through one or more tubes of UV light sources 706, 716. For example, such baffles 710, air channels, and/or air flow pathways may enable fan 714 to push the air flow through U-shaped tubes of the UV light sources 706, 716.

In this example, the system 700 includes the filter 704. The filter 704 may include an antimicrobial, carbon, activated carbon, cotton, disposable, electrostatic, fiberglass, foam, high efficiency particulate air (HEPA), impingement, mesh, odor reducing, polyester, pleated, replaceable, treated, washable, zeolite, another suitable type of filter, etc. In some examples, the filter 704 may include more than one type of filter. For instance, the filter 704 may include a HEPA filter and an activated carbon filter. In another example, the filter 704 may include a zeolite filter and an activated carbon filter. In one example, the filter 704 may include a HEPA, zeolite, and an activated carbon filter. In some examples, the filter 704 may trap air contaminants that remain after exposure to germicidal UV wavelengths produced by UV light sources 706, 716.

In this example, the power supply 712 provides clean power for consistent illumination of UV light sources 706, 716. Clean power provides a sufficiently stable amount of voltage that includes minimal variation, spikes, drops, abnormalities, etc. in a specified amplitude and frequency of the amount of voltage that is required to operate various components of system 700.

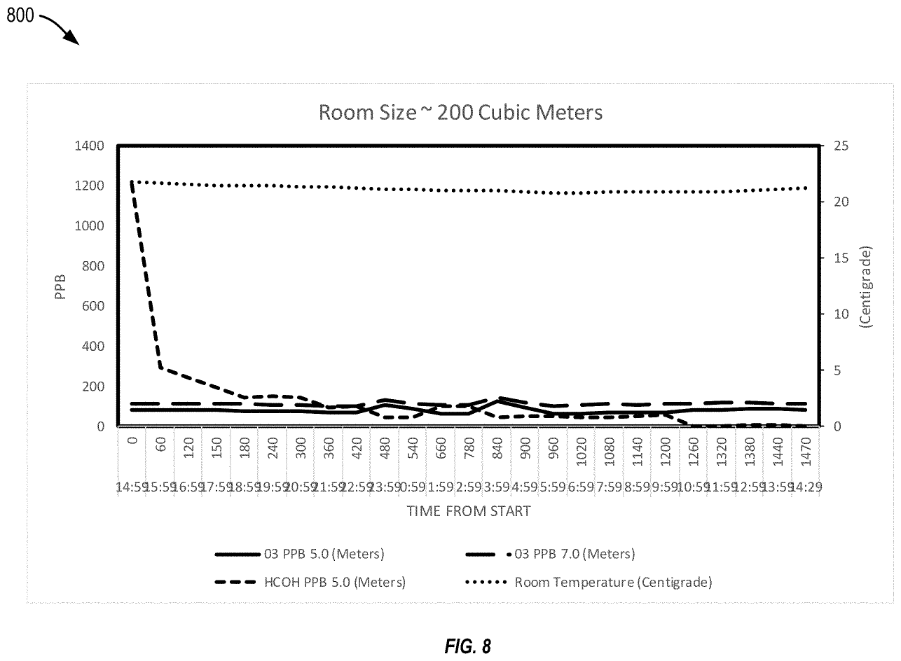

Referring now to FIG. 8, FIG. 8 shows a graph 800 of test results using a system for UV treatment of ambient indoor contaminants according to this disclosure over time. The graph 800 shows test results for a room with a system for UV treatment of ambient indoor contaminants. In this example, a UV treatment system 108 that was tested included a UV light tube that was approximately 1 meter in total length, end-to-end. The UV light tube included two sections that produced two frequency ranges, where a substantially longer, e.g., 5-10× longer, portion included a higher frequency range than a shorter portion of the UV light tube. The test results include data obtained from one or more temperature, ozone (O3), and formaldehyde (HCOH) sensors. Further, the graph 800 includes a leftmost y-axis that shows contaminant concentration in parts per billion (ppb), a rightmost y-axis that shows temperature in degrees centigrade (i.e., Celsius), and an x-axis that shows a change in contaminants and temperature over time (e.g., measured in minutes). In this example, the test results shown in graph 800 correspond to a room size that is approximately 200 cubic meters.

The graph 800 shows test results for the room that was measured at a room temperature of approximately 22 degrees centigrade (approximately 72 degrees Fahrenheit). Ozone sensor data was obtained at a relative distance of 5 and 7 meters, while formaldehyde data was obtained at a relative distance of 5 meters. In this example, the sensed amount of ambient air contaminant of formaldehyde was reduced from approximately 1100 ppb to 300 ppb in a first 60 minutes of operation by the UV treatment system. Further, over the course of the measured 24.5 hours, the sensed amount of formaldehyde was reduced to substantially zero, while the sensed amount of ozone remain substantially unchanged.

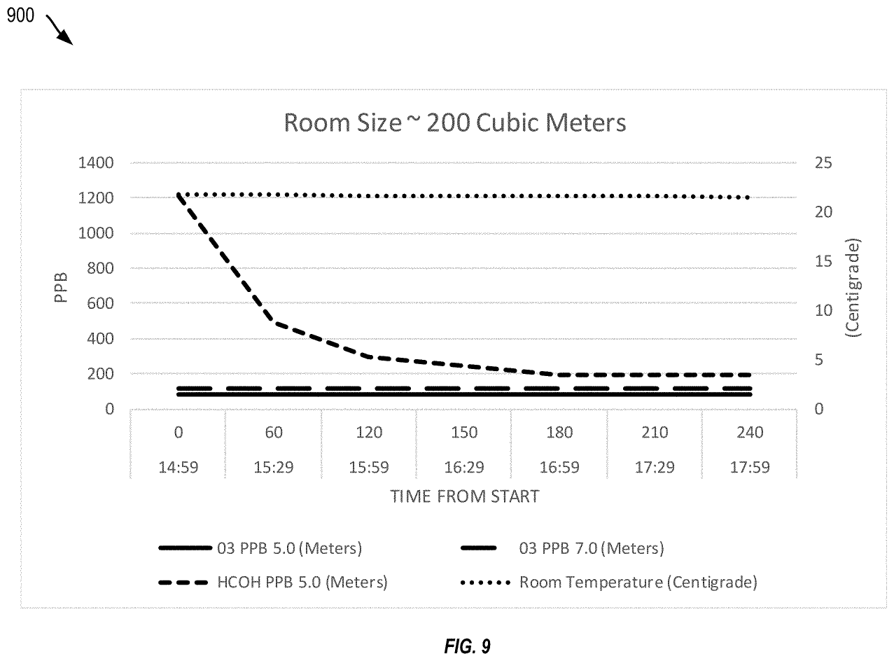

Referring now to FIG. 9, FIG. 9 shows another graph 900 of test results using a system for UV treatment of ambient indoor contaminants over time. The graph 900 shows test results for a room with a system for UV treatment of ambient indoor contaminants. In this example, a UV treatment system 108 that was tested is substantially similar to the tested UV treatment system 108 described with regard to FIG. 8. The test results include data obtained from one or more temperature, ozone (03), and formaldehyde (HCOH) sensors. The graph 900 includes a leftmost y-axis shown in ppb, a rightmost y-axis shown in degrees centigrade, and a x-axis shown in minutes. The test results shown in graph 900 correspond to a room size of approximately 200 cubic meters.

In this example, the graph 900 shows that different concentrations of VOCs, bacteria, viruses, etc. may react differently when treated by the UV treatment system 108. For instance, the 8-hour test shows a somewhat slower disinfection rate for formaldehyde. In some examples, the UV treatment system 108 may have a different efficacy for a first air contaminant (e.g., a 99.92% efficacy against H1N1 swine flu) and another efficacy for a second contaminant (e.g., a 92.86% efficacy against Salmonella) over an 8-hour duration.

In some examples, an overall size of the UV treatment system 108, which corresponds to an overall size of its UV light source 116, may be optimized for a particular sized room. For instance, the UV treatment system 108 tested may be optimal for a room size of approximately 100-200 cubic meters. But in some examples, a smaller version of the UV treatment system 108 (and UV light source 116) may be appropriate for a smaller space (e.g., 25-100 cubic meters). Likewise, a larger version of the UV treatment system 108 (and UV light source 116) may be appropriate for a larger space (e.g., 200-400 cubic meters). And in some examples, the UV treatment system 108 may be optimized by balancing a desired disinfection rate and ambient noise level.

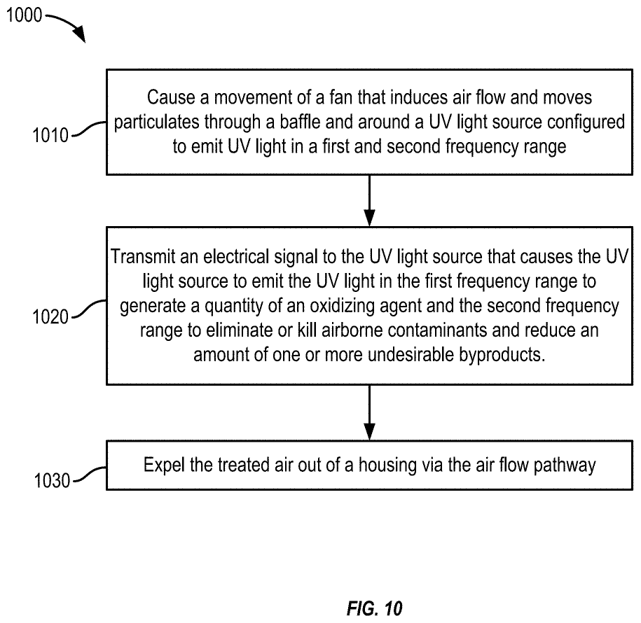

Referring now to FIG. 10, FIG. 10 illustrates an example method 1000 for enabling ultraviolet treatment of ambient indoor contaminants. The example method 1000 will be discussed with regard to the system 100 shown in FIG. 1, however, it should be appreciated that example methods according to this disclosure may be employed with any suitable system according to this disclosure.

At block 1010, UV treatment system 108 causes a movement of a fan (e.g., fan 114) that induces air flow and moves ambient indoor contaminants from an environment through an inlet in a housing. The air passes one or more baffles and a UV light source (e.g., UV light source 116) configured to emit UV light in a first and second frequency range. The baffle may be any type of baffle described herein. The baffle can slow an air flow induced by fan 114 and reduce an air velocity within UV treatment system 708. The reduced air velocity may provide an increased amount of contact time that the induced air flow is exposed to germicidal UV light produced by UV light source 116.

At block 1020, the UV treatment system 108 transmits an electrical signal to the UV light source 116 that causes the UV light source to emit the UV light in the first frequency range to generate a quantity of an oxidizing agent and the second frequency range to eliminate or kill indoor contaminants and reduces an amount of undesirable byproducts. For example, the UV light may reduce the amount of undesirable byproducts by degrading at least a portion of the quantity of the oxidizing agent. The UV light source 116 may be illuminated according to any of the techniques described herein.

At block 1030, the UV treatment system 108 expels the treated air out of the housing via the air flow pathway. For example, the UV treatment system 108 can use the fan 114 to expel the treated air out of the housing and into a predefined space according to any of the techniques described herein.

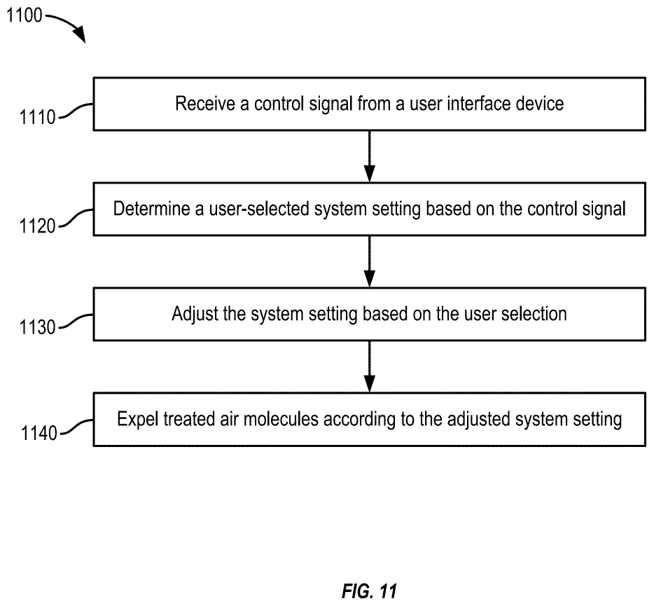

Referring now to FIG. 11, FIG. 11 illustrates an example method 1100 for UV treatment of ambient indoor contaminants. The example method 1100 will be discussed with regard to the system 100 shown in FIG. 1, however, it should be appreciated that example methods according to this disclosure may be employed with any suitable system according to this disclosure.

At block 1110, the UV treatment system 108 receives a control signal from a user interface device (e.g., client device 104). In this example, the client device 104 includes a user interface that enables the client device 104 to send the control signal to the UV treatment system 108. The client device may include any client device or interface device described above, with regard to FIGS. 1-8.

At block 1120, the UV treatment system 108 determines a user-selected system setting based on the control signal from block 1110. For example, after receiving the control signal, the UV treatment system 108 may execute settings module 118 to determine the user-selected system setting. In some examples, the settings module 118 may determine the user-selected system setting includes any of the settings described herein.

At block 1130, the UV treatment system 108 adjusts the system setting based on the user selection. For example, the UV treatment system may change an amount of air flow, air circulation pattern, air velocity, blower or fan speed, directionality, duty cycle, filter control, lighting, noise level, notification, occupancy, on/off control, room setting, setup, timer, unit of measurement, ventilation, volume control, etc.

At block 1140, the UV treatment system 108 expels treated air molecules according to the adjusted system setting from block 1130. For example, the UV treatment system 108 can use the fan 114 to expel the treated air out of the housing and into a predefined space, based on the user selection, according to any of the techniques described herein.

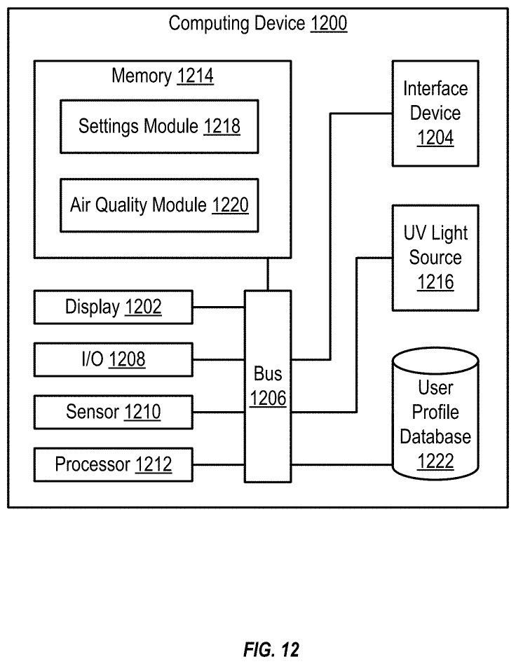

Referring now to FIG. 12, FIG. 12 shows an example computing device 1200 for a system for UV treatment of ambient indoor contaminants. The computing device 1200 includes a processor 1212 in communication with other hardware via a bus 1206. A memory 1214, which can be any suitable tangible (and non-transitory) computer-readable medium such as random access memory (“RAM”), read-only memory (“ROM”), erasable and electronically programmable read-only memory (“EEPROMs”), or the like, embodies program components (e.g., settings module 1218, air quality module 1220, etc.) that configure operation of the computing device 1200. In the embodiment shown, computing device 1200 further includes a display 1202, interface device 1204, input/output (“I/O”) components 1208, sensor 1210, UV light source 1216, and user profile database 1222.

It should be appreciated that computing device 1200 may also include additional processors, additional storage, and a computer-readable medium (not shown). The processor(s) 1212 may execute additional computer-executable program instructions stored in memory. Such processors may include a microprocessor, digital signal processor, application-specific integrated circuit, field programmable gate arrays, programmable interrupt controllers, programmable logic devices, programmable read-only memories, electronically programmable read-only memories, or other similar devices.

The computing device 1200 also includes I/O components 1208. In some examples, the I/O components 1208 may enable communications using one or more networks, including a local area network (“LAN”); wide area network (“WAN”), such as the Internet; metropolitan area network (“MAN”); point-to-point or peer-to-peer connection; etc. Communication with other devices may use any suitable networking protocol. For example, one suitable networking protocol may include the Internet Protocol (“IP”), Transmission Control Protocol (“TCP”), User Datagram Protocol (“UDP”), Bluetooth, or combinations thereof, such as TCP/IP or UDP/IP.

The methods, devices, and systems discussed above are examples. Various configurations may omit, substitute, or add various procedures or components. For example, in alternative configurations, the methods may be performed in a different order. In another example, the methods may be performed with fewer steps, more steps, or in combination. In addition, certain configurations may be combined in various configurations. As technology evolves, many of the elements are examples and do not limit the scope of the disclosure or claims.

While some examples of methods and systems herein are described in terms of software executing on various machines, the methods and systems may also be implemented as specifically-configured hardware, such as field-programmable gate array (FPGA) specifically to execute the various methods according to this disclosure. For example, examples can be implemented in digital electronic circuitry, or in computer hardware, firmware, software, or in a combination thereof. In one example, a device may include a processor or processors. The processor comprises a computer-readable medium, such as a random access memory (RAM) coupled to the processor. The processor executes computer-executable program instructions stored in memory, such as executing one or more computer programs. Such processors may comprise a microprocessor, a digital signal processor (DSP), an application-specific integrated circuit (ASIC), field programmable gate arrays (FPGAs), and state machines. Such processors may further comprise programmable electronic devices such as PLCs, programmable interrupt controllers (PICs), programmable logic devices (PLDs), programmable read-only memories (PROMs), electronically programmable read-only memories (EPROMs or EEPROMs), or similar devices.

Such processors may comprise, or may be in communication with, media, for example non-transitory computer-readable media, that may store processor-executable instructions that, when executed by the processor, cause the processor to perform methods according to this disclosure as carried out, or assisted, by a processor. Examples of non-transitory computer-readable medium may include, but are not limited to, an electronic, optical, magnetic, or other storage device capable of providing a processor, such as a processor in a server with processor-executable instructions. Other examples of non-transitory computer-readable media include, but are not limited to, a floppy disk, CD-ROM, magnetic disk, memory chip, ROM, RAM, ASIC, configured processor, all optical media, all magnetic tape or other magnetic media, or any other medium from which a computer processor can read. The processor, and the processing, described may be in one or more structures, and may be dispersed through one or more structures. The processor may comprise code to carry out methods (or parts of methods) according to this disclosure.

The foregoing description of some examples has been presented only for the purpose of illustration and description and is not intended to be exhaustive or to limit the disclosure to the precise forms disclosed. Numerous modifications and adaptations thereof will be apparent to those skilled in the art without departing from the spirit and scope of the disclosure.

Reference herein to an example or implementation means that a particular feature, structure, operation, or other characteristic described in connection with the example may be included in at least one implementation of the disclosure. The disclosure is not restricted to the particular examples or implementations described as such. The appearance of the phrases “in one example,” “in an example,” “in one implementation,” or “in an implementation,” or variations of the same in various places in the specification does not necessarily refer to the same example or implementation. Any particular feature, structure, operation, or other characteristic described in this specification in relation to one example or implementation may be combined with other features, structures, operations, or other characteristics described in respect of any other example or implementation.

Use herein of the word “or” is intended to cover inclusive and exclusive OR conditions. In other words, A or B or C includes any or all of the following alternative combinations as appropriate for a particular usage: A alone; B alone; C alone; A and B only; A and C only; B and C only; and A and B and C.

Specific details are given in the description to provide a thorough understanding of example configurations (including implementations). However, configurations may be practiced without these specific details. For example, well-known circuits, processes, algorithms, structures, and techniques have been shown without unnecessary detail to avoid obscuring the configurations. This description provides example configurations only, and does not limit the scope, applicability, or configurations of the claims. Rather, the preceding description of configurations will provide those skilled in the art with an enabling description for implementing described techniques. Various changes may be made in function and arrangement of elements without departing from the spirit or scope of the disclosure.

That which is claimed is:

1. A ultraviolet (UV) treatment system comprising:
a housing;
a fan positioned within the housing, the fan configured to induce air flow into and out of the housing through an air flow pathway;
a UV light source positioned within the housing and the air flow pathway, the UV light source configured to emit UV light in a first frequency range from a first portion of the UV light source and UV light in a second frequency range from a second portion of the UV light source, and wherein the first frequency range is different from the second frequency range;
a baffle positioned within the housing and within the air flow pathway, the baffle configured to slow the air flow induced by the fan to increase exposure time of the air flow to the UV light source; and
wherein the UV treatment system is configured to:
cause a movement of the fan that induces the air flow and moves contaminants through the baffle and around the UV light source;
transmit an electrical signal to the UV light source to cause the UV light source to emit the UV light in the first frequency range to generate a quantity of an oxidizing agent and in the second frequency range to eliminate or kill contaminants and reduce at least a portion of one or more undesirable byproducts; and
expel the treated air out of the housing via the air flow pathway.

2. The UV treatment system of claim 1, wherein the UV treatment system is wall-mounted or floor mounted.

3. The UV treatment system of claim 1, wherein the fan is a centrifugal fan.

4. The UV treatment system of claim 1, wherein the first frequency range does not overlap with the second frequency range.

5. The UV treatment system of claim 1, wherein the UV light source comprises:
a U-shaped tube having at least two segments;
a first gas disposed in a first segment of the at least two segments, the first gas configured to generate the UV light in the first frequency range when energized; and
a second gas disposed in a second segment of the at least two segments, the second gas configured to generate the UV light in the second frequency range when energized, and wherein the second gas is different from the first gas.

6. The UV treatment system of claim 5, wherein the UV light in the second frequency range is selected to reduce an amount of formaldehyde, carbon dioxide concentration, volatile organic compounds, bacteria, or viruses.

7. The UV treatment system of claim 1, further comprising:
a non-transitory computer-readable medium; and
a processor in communication with the non-transitory computer-readable medium, the processor configured to execute instructions stored in the non-transitory computer-readable medium to:
transmit a first signal to control the movement of the fan; and
transmit a second signal to cause the electrical signal to be output to the UV light source to cause the UV light source to emit the UV light in the first and second frequency ranges.

8. The UV treatment system of claim 7, wherein the processor configured is further to execute instructions stored in the non-transitory computer-readable medium to:
receive a control signal from a remote computing device; and
change a user-selected setting based on the control signal.

9. The UV treatment system of claim 8, wherein the remote computing device comprises a smartphone, tablet, laptop, or control panel.

10. The UV treatment system of claim 8, wherein the user-selected setting comprises one or more of an on/off command, start/stop function, timer, fan speed, or circulation pattern.

11. The UV treatment system of claim 7, further comprising a sensor, and wherein the processor further configured to execute instructions stored in the non-transitory computer-readable medium to:
receive a sensor signal from the sensor, wherein the sensor signal indicates an environmental condition; and
adjust a control setting based on the environmental condition.

12. The UV treatment system of claim 11, wherein the environmental condition comprises a measurement of ambient air quality.

13. A method comprising:
inducing, by a fan positioned within a housing, an air flow into the housing and through an air flow pathway to an outlet from the housing, wherein:
a UV light source is positioned within the housing and the air flow pathway, the UV light source configured to emit UV light into the air flow, and
a baffle is positioned within the housing and the air flow pathway, the baffle configured to slow the air flow past the UV light source;
emitting, by the UV light source, UV light in a first frequency range to generate a quantity of an oxidizing agent and in the second frequency range to eliminate or kill contaminants and reduce at least a portion of one or more undesirable byproducts, and wherein the first frequency range is different from the second frequency range; and
expelling, by the fan, the treated air out of the housing via the air flow pathway and the outlet from the housing.

14. The method of claim 13, wherein the first frequency range does not overlap with the second frequency range.

15. The method of claim 13, wherein the UV light source comprises:
a U-shaped tube having at least two segments;
a first gas disposed in a first segment of the at least two segments, the first gas configured to generate the UV light in the first frequency range when energized; and
a second gas disposed in a second segment of the at least two segments, the second gas configured to generate the UV light in the second frequency range when energized, and wherein the second gas is different from the first gas.

16. The method of claim 15, wherein the UV light in the second frequency range is selected to reduce an amount of formaldehyde, carbon dioxide concentration, volatile organic compounds, bacteria, or viruses.

17. The method of claim 13, further comprising:
transmitting, by a processor, a first signal to control the movement of the fan; and
transmitting, by the processor, a second signal to cause the UV light source to emit the UV light in the first and second frequency ranges.

18. The method of claim 17, further comprising:
receiving, by the processor, a control signal from a remote computing device, wherein the remote computing device comprises a smartphone, tablet, laptop, or control panel; and
changing, by the processor, a user-selected setting based on the control signal, wherein the user-selected setting comprises changing one or more of an on/off command, start/stop function, timer, fan speed, or circulation pattern.

19. The method of claim 18, further comprising:
receiving, by a processor, a sensor signal from a sensor, wherein the sensor signal indicates an environmental condition, wherein the environmental condition comprises a measurement of ambient air quality; and
adjusting, by the processor, a control setting based on the environmental condition.

20. A non-transitory computer-readable medium comprising program code executable by a processor to cause the processor to:
transmit a first signal to control a movement of a fan of a UV treatment system, the UV treatment system comprising:
a housing;
the fan positioned within the housing, the fan configured to induce air flow into and out of the housing through an air flow pathway;
a UV light source positioned within the housing and the air flow pathway, the UV light source configured to emit UV light in a first frequency range from a first portion of the UV light source and UV light in a second frequency range from a second portion of the UV light source, and wherein the first frequency range is different from the second frequency range; and
a baffle positioned within the housing and within the air flow pathway, the baffle configured to slow the air flow induced by the fan to increase exposure time of the air flow to the UV light source; and
transmit a second signal to cause the UV light source to emit the UV light in the first and second frequency ranges, wherein UV light in the first frequency range is configured to generate a quantity of an oxidizing agent and UV light in the second frequency range is configured to eliminate or kill contaminants and reduce at least a portion of one or more undesirable byproducts to treat the air in the air flow.
